(19) United States
(12) Patent Application Publication
(10) Pub. No.: US 2014/0335503 A1
(43) Pub. Date: Nov. 13, 2014

(54) METHODS FOR ASCERTAINING THE HISTORY OF HEMATOLOGICAL CONDITIONS OF A HUMAN FROM A SINGLE BLOOD SAMPLE

(71) Applicant: Alex M. SAUNDERS, San Carlos, CA (US)

(72) Inventor: Alex M. SAUNDERS, San Carlos, CA (US)

(21) Appl. No.: 14/273,217

(22) Filed: May 8, 2014

Related U.S. Application Data
(60) Provisional application No. 61/822,495, filed on May 13, 2013.

(51) Int. Cl. G01N 33/80; G01N 33/72; G01N 33/74; C12Q 1/48

(52) U.S. Cl. CPC G01N 33/80; C12Q 1/485; G01N 33/721; G01N 33/746; G01N 2800/224; G06F 19/28
USPC 435/2; 702/19

(57) ABSTRACT

A method for analysis of red blood cell production dynamics is disclosed. The method comprises: (ai) separating RBCs in the blood sample into ordered subsets of RBCs based on relative age-related physical parameter of the RBCs, or (aii) obtaining a pair of estimates simultaneously, the pair of the estimates comprising a first estimate and a second estimate, the first estimate being a measurement of the size of individual RBCs, the second estimate being a measurement of the activity or concentration of the biomarker, sorting the pair into ordered subsets of data based on the first estimate to obtain paired estimates; (b) generating a mathematical pattern or a graphical pattern based on the paired estimates or the ordered subsets of data; and (c) comparing the mathematical pattern or the graphical pattern with established patterns to determine the RBC production dynamics in the person.

### REFERENCE TO RELATED APPLICATION

The present application claims the priority to U.S. Provisional Application Ser. No. 61/822,495, filed May 13, 2013, which is herein incorporated by reference in its entirety.

### FIELD OF THE INVENTION

The invention relates to a method of determining Red Blood Cell production dynamics according to a physical parameter related to age and measuring a biochemical parameter of aging in the subsets.

### BACKGROUND OF THE INVENTION

Red blood cells (RBCs) normally circulate in human blood for 120 days before they are removed. The selection process for RBC removal from circulation is not well understood. Aging of RBC from the time they enter circulation as reticulocytes to the time when they leave circulation has been studied by a number of methods. For example, the presence of a minimum amount of RNA in the cells as they enter circulation is identified by stains that show up the RNA as a meshwork. Reticulum is another name for a meshwork, and hence these cells are called “reticulocytes”. Hence the number of reticulocytes in circulation is an estimate of the very youngest cells in circulation. These cells usually have disappeared by the second day in circulation, hence the normal number of reticulocytes in circulation is calculated as 1/120=0.008125, or approximately 0.8% of total RBCs.

An increased number of reticulocytes in circulation is a strong indicator of increased RBCs production activity. With some rare exceptions the increased production is a consequence of increased demand and is mediated through an internal body stimulus in which erythropoietin is the biochemical signal to the RBC-manufacturing precursor cells. Hence one of the main exceptions to reticulocytes being a sign of internal stimulus for production is the exogenous supply or treatment with erythropoietin.

As aforementioned, reticulocytes disappear from circulation in less than two days after the RBC enters circulation. By use of standard studies all history of a recent demand for RBC production increase is lost when the reticulocyte count returns to normal. However, in the classification of hematological conditions, and also in evaluation, of hematological therapy, it would often be of value to know the recent history of increased production of RBC. Similarly, it would be important to reliably determine when an athlete has built up an excess of RBC by abusing erythropoietin. Obviously the athlete would not come to his or her athletic event with an increased reticulocyte count. Hence knowledge of RBCs that were recently reticulocytes would be a powerful adjunct to athletic monitoring for abused substances.

Hematologists have on occasion used extraordinary methods of evaluating RBC aging. For example, labeling the newly made cells with a pulse of radioactive iron enables monitoring of a cohort of RBC throughout its lifetime. When an RBC sample so labeled is centrifuged at high speed, the radioactive label is first observed in the layer of lightest cells on the top of the cell pack. Two months after the pulse labeling, the radioactive cells are found in the middle layers of the cell stack. At four months after the cohort of cells is labeled the radioactive cells are observed at the bottom of the cell stack after high speed centrifugation. (E R Borun et al “Distribution of Fe⁵⁹ tagged human erythrocytes in centrifuged specimens as a function of cell age. J. Clin. Invest 36: 676 1957) From such experiments it has been determined that relative RBC age is reliably determined by the relative density of RBC compared to each other.

The increase of relative density of RBC during aging has been shown to relate to the volume of the cell. The observation is that as RBC age there is less volume, and yet the total hemoglobin remains constant. Hence the old cells have a higher internal concentration of hemoglobin, which is a very dense (iron containing) molecule, and are in truth more dense than younger cells. The density-age technique is thereby confirmed. (AM Saunders et al “Fetal Cells from Maternal Blood: design of a Product Laboratory Hematology 3: 282 1997.)

The measure of relative RBC size is already used in clinical hematology devices. The test is named RDW, or Red cell Distribution Width. A wider RDW will be found during changes in size of RBC Age interpretation has not been suggested for RDW.

It has been known for a long time that certain of the enzymes inside RBC lose activity over its lifetime. This makes sense because RBC have lost the machinery to make new proteins and proteins that are damaged cannot be replaced. The enzyme activity is highest in reticulocytes, and is gradually lost over time. Hence a determination of relative enzyme activity may provide an indication of the number of cells that were recently reticulocytes, and would expand the ability to diagnose recent hematological events. A combination of the independent parameters or enzyme activity and RBC size would provide an additional understanding of RBC dynamics and assist in the differentiation of a variety of hematological conditions.

In a prior U.S. patent (U.S. Pat. No. 5,550,060) by the same inventor methods were described for separation of RBC by age for the purpose of time resolved analysis of glycated hemoglobin. The age of cells was confirmed by the gradual decrease over time of the enzyme Pyruvate Kinase (PK). Thus the analysis of both Pyruvate Kinase and glyco-hemoglobin in the same density centrifuged samples served to validate the time resolved glycation of hemoglobin. However the patterns of Pyruvate kinase activity were not examined in detail. Those patterns have now been correlated with certain hematological conditions and added value is observed in relating PK analysis with relative cell age for screening, diagnosing or monitoring hematological conditions.

It is also recognized that RBC enzymes which are known to degrade during the natural life of the cell also continue to decrease activity during refrigerated storage. This suggests that certain irregular patterns would appear in detailed patient blood analysis after blood transfusion. Even if the transfused cells are of the same person's blood (an auto-transfusion) the distorted pattern would appear.

### SUMMARY OF THE INVENTION

Detecting auto-transfusion at athletic events by relating the transfusion to a specific pattern of degradation of the estimate of enzyme activity is one feature of the present invention. In one aspect, the invention relates to a method for analysis of Red Blood Cell production dynamics, which comprises:
a) obtaining a blood sample from a person;
b1) separating RBCs in the blood sample into ordered subsets of RBCs based on relative age-related physical parameter of the RBCs to obtain a first estimate of relative RBC age for each of the subsets of the RBCs, and obtaining a second estimate of the activity or concentration of a biomarker that degrades with RBC age within the subsets of the RBCs, and pairing the first estimate and the second estimate for each of the subsets to obtain paired estimates; or
b2) obtaining a pair of estimates simultaneously, the pair of the estimates comprising a first estimate and a second estimate, the first estimate being a measurement of the size of individual RBCs, the second estimate being, a measurement of the activity or concentration of the biomarker, sorting the pair into ordered subsets of data based on the first estimate to obtain paired estimates;
c) generating a mathematical pattern or a graphical pattern based on the paired estimates of the ordered subsets of data; and
d) comparing the mathematical pattern or the graphical pattern with established patterns to determine the RBC production dynamics in the person.

In one embodiment of the invention, the separating step is performed by density centrifugation.

In another embodiment of the invention, the one or more enzymes is at least one selected from the group consisting of pyruvate kinase or hexokinase.

In one embodiment of the invention, the aforementioned method further comprises determining that the person has a particular condition that affects the RBC production dynamics thereof by comparing the generated pattern with established patterns that reflect the particular condition.

In one embodiment of the invention, the aforementioned method further comprises interpreting that the person is rapidly producing RBCs immediately before the sample is obtained when the graphical pattern shows the second estimate for a young age RBC subset is higher than the remaining ordered subsets.

In one embodiment of the invention, the aforementioned method further comprises interpreting that the person is rapidly producing RBCs before the sample is obtained when the graphical pattern shows the second estimate for a relatively older age RBC subset is higher than the remaining ordered subsets.

In one embodiment of the invention, the aforementioned method further comprises interpreting that the person has or recently had a high blood level of erythropoietin as compared with a normal person when the graphical pattern shows the second estimates remains high.

In one embodiment of the invention, the aforementioned method further comprises interpreting that the person has an abnormal hematological condition when the mathematical pattern falls outside the range of a normal statistical population distribution of mathematical patterns that are generated from a collection of slopes, intercepts and/or correlation coefficients of the paired estimates.

In one embodiment of the invention, the step of obtaining the pair of estimates simultaneously is performed by flow cytometry.

In one embodiment of the invention, the aforementioned method further comprises interpreting that the person receives an RBC supplementation before the blood sample is obtained when the graphical pattern exhibits more than one discontinuity and the second estimates remains low or normal.

In one embodiment of the invention, the RBC supplementation is autologous blood transfusion.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 is a graph of pyruvate kinase measurements of density gradient fractions of normal blood.

FIG. 2 is a graph of pyruvate kinase measurements of density gradient fractions of a patient with a 10% blood loss in the last 5 days.

FIG. 3 is a graph of pyruvate kinase measurements of density gradient fractions of a patient with chronic autoimmune hemolytic anemia

FIG. 4 is a graph of pyruvate kinase measurements of density gradient fractions of a patient responding to severe blood loss over time, and demonstrates a severe discontinuity in the curve

FIG. 5 is a histogram of the distribution of intercepts for pyruvate kinase curves in 117 patients.

FIG. 6 is a graph of the predicted activity of pyruvate kinase in aging red blood cells using a half life of 29 days.

FIG. 7 is a histogram of the distribution of correlation coefficients for pyruvate kinase curves in 117 patients.

FIG. 8 is a histogram of the distribution of slopes for pyruvate kinase curves in 117 patients.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification. Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “relative RBC age” is the age of RBC in a sample as compared to other RBC in the same sample. The emphasis is on relative age since the analytical procedure cannot discern the absolute age in days but only the order of cell age within the estimate used. For example, in the normal condition the oldest cells are approximately 120 days old. In a condition of repetitive cell loss due to hemorrhage or hemolysis there are very few RBC that survive to 120 days.

The term “red blood cell production dynamics” general means the complete process in the human body for making of red blood cells and permitting them to go into circulation. The process includes control stimuli acting upon the cells that divide and become Red Blood Cells and also includes specific responses to the stimuli. In theory all parts of the process are quantitatively related and continuously changing in terms of stimulus and response. That is why dynamic is the appropriate reference word. In practice, however, not all the variables of stimulus and response are easily quantified. In medical practice the immediate response is often measured by the proportion of Red Blood Cells that are reticulocytes. The present invention extends the ability to quantify the response beyond the time of disappearance of RNA from the reticulocytes, and therefore broadens the scope of understanding Red Blood Cell production dynamics.

The term “RBC dynamics” refers to the entire process that include, but not limited to, stimulations of RBC production, RBC production, RBC circulation and aging within a human body.

The term “estimate of red blood cell age” means evaluating a feature of the Red Blood Cell that has a known relationship with the time that the Red Blood Cell has been in circulation in a person.

The term “density separation” means a method of providing an ordered series of age related subsamples for further analysis. Density changes with time of RBC in circulation independently of changes that may occur within the RBC. Density is one estimate of RBC relative age.

The term “cytometric size analysis” means one of several parameters to be applied by flow cytometry or other analysis, whereby individual cell measurements are placed in sequential order of relative age after arriving at the cytometry sensor randomly, while other parameters may be additionally applied to the same cell. These further parameters may, for example, involve staining cells quantitatively for their activity of certain enzyme or enzymes. Cytometric size analysis is another estimate of RBC relative age.

The term “correlative pattern” refers to quantitative or graphical methods of extracting novel information from the combined relative age and activity measurements of individual cells by cytometry, or by the analysis of age related subsets.

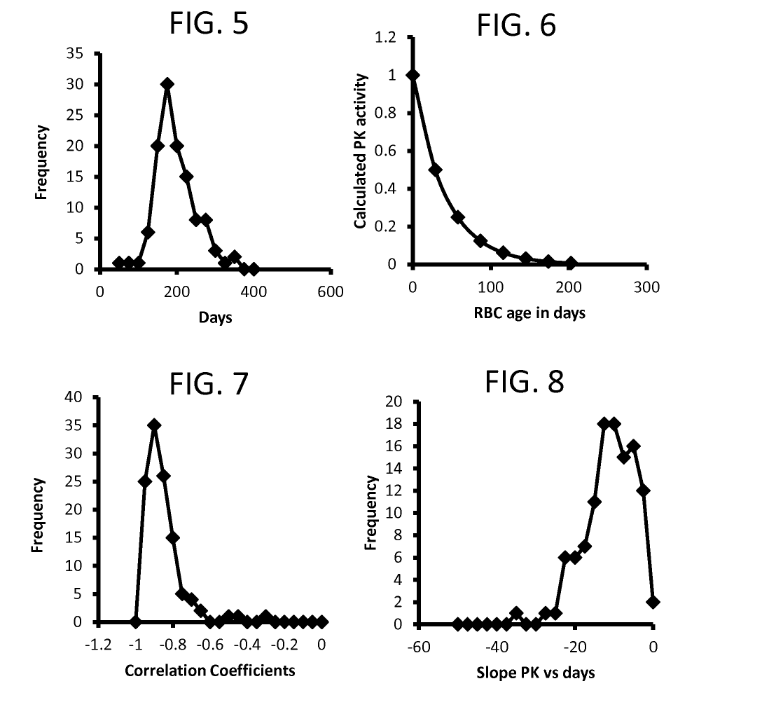

The term “a mathematical pattern” includes, but not limited to, a slope, an intercept, and a correlation coefficient. A statistical population distribution of mathematical patterns is illustrated in FIGS. 5. 7, and 8, each of which shows a normal distribution and an abnormal (outliers) distribution.

The term “paired estimates” refers to a series of ordered measurements or ordered subsets (which are numbers reflecting relative age of RBCs).

The term “an established pattern” means a pattern drawn from a large population and/or collection of patterns that are obtained from individual persons and correlated with particular, known conditions. When a person has a pattern similar to one of the established patterns, it can be interpreted that the person has the condition of the established pattern.

The present invention relates to methods of analysis and correlation of results of RBC enzyme activity with the independently measured age of the cell and how these relate to hematological conditions. The present invention, among other advantages, provides a reliable method of identifying RBC that recently entered circulation.

One variation of the invention is by providing density related fractions of RBC from a sample. The method employed is precisely that of U.S. Pat. No. 5,550,060, which is here referenced in its Entirety. According to the prior art the density separation may be enhanced by high speed centrifugation after treating the sample with the drug Chlorpromazine. This drug treatment affects the RBC membrane decreasing the resistance of individual cells as they slide past each other while responding to their relative density compared to other cells. Each cell therefore moves easily past other cells while to finding its neutral buoyant density. At equilibrium, each cell will therefore have more dense cells below it and less dense cells above it. The centrifugation is stopped when equilibrium is obtained. Then a series of age ordered fractions is separated from the resultant cell stack. Usually there are 10 fractions in a sample analysis. Such ordered fractions may then be analyzed for the total hemoglobin, and for any chemical characteristic such as an enzyme activity or a degree of glycation of the hemoglobin molecule

Analysis of the subsets leads to the formation of patterns related to the relative age of RBC, coordinated with at least one other parameter or enzyme activity. It has been found, for example, that in hematologically normal individuals the first fraction contains a relatively high activity of Pyruvate Kinase (PK) because in addition to RBC, this fraction contains all the white blood cells which have much higher PK activity. The same first fraction also contains the youngest RBC, or reticulocytes. All the other ordered sub-fractions will display an ordered decrease in PK activity. In the case of a normal the decrease is linearly ordered as compared to the fraction series as will be demonstrated in the examples.

The method of density centrifugation used in the invention is known to be far more efficient in separating RBC according to age than the density gradient centrifugation used by the workers who identified the trends of enzyme and surface marker degradation in red blood cells. Density gradient fractions used by Camagna et al (Diabetes Vol 32, 1017, 1983) for example had reticulocytes in three out of their five fractions. Neither Camagna et al, nor Seaman et al, were able to provide data on individual person with sufficient resolution to produce reliable patterns of disease related conditions. The use of artificial gradients by Camagna and by Seaman limited their ability to obtain the required resolution. The present invention uses high speed centrifugation and chrlorpromazine without an artificial gradient to achieve the desired cell gradient. Novelty of the present invention is in the discovery of detailed patterns associated with hematological disorders, therapy and response to blood loss.

Flow cytometry is another means of obtaining the RBC age related information of the invention. The reason for cells becoming more dense with age is that the cells lose volume but retain hemoglobin. Change of volume is easily measured in flow cytometry by light scattering. Each individual cell is therefore measured both for its light scattering and for presence of an activity by histochemical staining. Examples of such activity include the enzyme, Pyruvate Kinase and the ability of red blood cell membrane to bind insulin. Both of these activities are known to decrease significantly during the aging of red blood cells (A Camagna, et al, Diabetes, Vol 32, page 1017, 1983). The paired measurements are segregated into bins (subsets) of constant number of cells according to relative size. The average of bins may then be treated in like manner to calculations made for the density method already described. A variety of other patterns, related to individual cell analysis may also be performed in the flow cytometry application. Since the general patterns necessary for understanding the invention arc similar in the density method and the flow cytometry method all the following examples are confined to the density separation technique. There is no intention to limit the invention thereby to the density procedure. This invention responds to the need for detailed analysis of Red Blood Cells (RBC) dynamics. Presently reticulocyte count as percent of all RBC indicates production of RBC in response to demand or to medication within the past 1 or 2 days. When the reticulocytes lose the RNA content and disappear, the dynamics are no longer accessible for analysis by standard methods, although the aging process obviously continues. The invention enables recording a history for the production of RBC beyond the one or two days when the reticulocytes are present. it is the equivalent of performing a reticulocyte count on a patient daily by only taking one patient sample. The invention also provides indications of inconsistency in the normal production of RBC so that the history of transfusion may be evaluated. Even the history of autologous transfusions may be discovered. Therefore the invention may be used both for diagnosis and for detection of blood augmentation in athletes.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein, however, in no way they, whether they are right or wrong, should limit the scope of the invention so long as the invention is practiced according to the invention without regard for any particular theory or scheme of action.

### Example 1

### An Example of a Procedure for the Invention with Normal Blood

The sample source is EDTA anticoagulated blood from a human person. 300 microL of well mixed blood are transferred to a 2 mm diameter polyethylene tube that is sealed at one end. The tube is centrifuged in a microhematocrit centrifuge at 15000 RPM. The tube is then sliced with a scalpel into 10 equal sized RBC segments. Each segment is placed in an individual micro centrifuge tube and all sub sample tubes are subsequently treated identically. The total hemoglobin (Hb) is determined and recorded for each tube by spectrophotometry at 415 nm. Pyruvate Kinase (PK) activity of each sub fraction determined by use of a commercial kit obtained from Sigma Chemicals, St Luis Mo. Paired results of Hb and PK are tabulated. The total hemoglobin is summed and each hemoglobin recalculated as a fraction of the total. In the tabulation these hemoglobins are accumulated such that the final fraction is equal to a value of 1.0. The cumulative series is paired with the corresponding PK result. The results are plotted on a two dimensional chart with the relative age of cells on the horizontal axis. It should be noted that this cell age presentation is the reverse direction to calendar time.

Similar patient patterns and population relationships are expected by flow cytometric analysis. The invention incorporates the features of flow cytometry that may be derived from the same principles already expressed in the present invention.

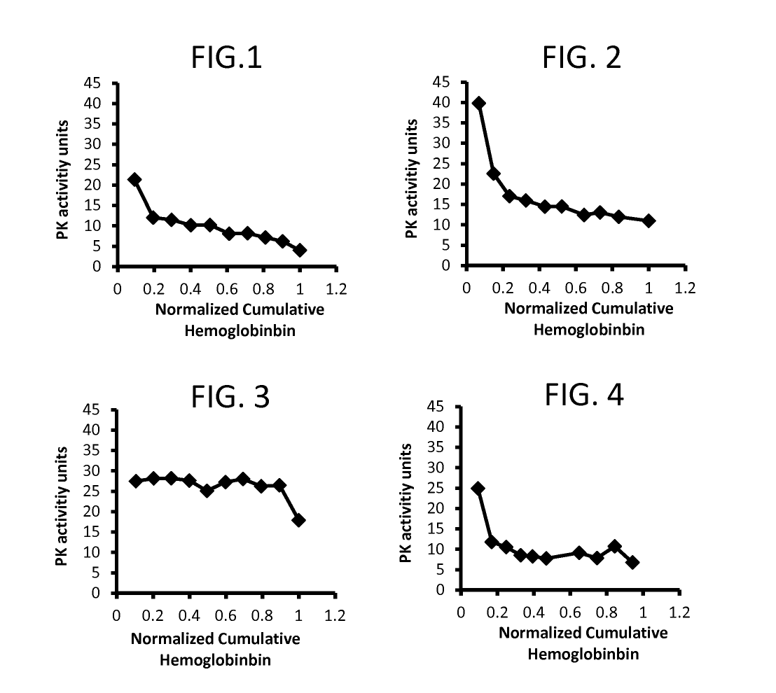

FIG. 1 contains an example of such a pattern. Features of interest in this example should be noted. With the exception of the first point (on the left of the chart) all the points form a straight line. Characteristics of that line of points may be summarized in mathematical terms. A correlation coefficient is the measure of the straightness of the line. If the line is descending the correlation will be negative. Closeness to −1.0 would indicate a better correlation than a lower negative number. In the presence of discontinuities of the line there would be a poorer correlation. The line also has a slope. A Steep slope would indicate a more rapid degradation of the Pyruvate Kinase activity. The position on the horizontal axis where the line would cross if it were further projected is known as the intercept. In the format of FIG. 1 the intercept would indicate the relative age where all the PK activity had disappeared. It may be translated to Red Blood Cell age by a multiplier of 120. According to prior art PK has a half life of 29 days. Accordingly to prior art the intercept should calculate to a number close to 170 days. This will be tested in a later example. The reason for the first point being off the straight line is related to the presence of white blood cells, with high PK activity being always present in the first fraction. In addition in the present method all the reticulocytes are also present in the first fraction. Therefore the calculations of correlation, slope and intercept exclude the first fraction.

### Example 2

### Recent Blood Loss

Sample from a patient with mild recent blood loss, for example from surgery, is treated as in example 1. The results are similarly plotted and are shown in FIG, 2. The result is shown to have elevation of PK activity also in the second fraction. Notice that two fractions show a steep slope in the change of PK activity due to sudden replacement of a percentage of the whole blood volume.

### Example 3

### Chronic Autoimmune Hemolytic Anemia

Example 1 was repeated with a patient with a chronic autoimmune Hemolytic anemia and was shown to have the pattern depicted in FIG. 3. The continuous elevated PK activity for cells at most relative ages is clearly shown. However in this patient the relative ages of RBC do not accumulate to 120 days. All the cells are relatively young, as indicated by the continued high PK activity.

This patient was known to have an almost normal total hemoglobin level of 12.8 g/dL. This pattern may be recognized as relating to “Compensated hemolytic anemia.” The same pattern, however, would be observed with continuous application of erythropoietin from an exogenous source. This pattern gives superior information than is available in the prior art. Reticulocytes would be high, but only indicate the patient's response in the last 2 days.

### Example 4

### Severe Discontinuity in the Curve

The experiment of example 1 was again repeated on random samples with minimal clinical histories. A pattern of non linear degradation related to cell density or cell size may be expected when there is a history of transfusion or intermittent treatment for purpose of Red Blood Cell formation. Such a pattern was found in a relatively small series of examined patients. FIG. 4 is an example of severe blood loss in a patient with nutritional anemia with consequent stimulus and complete replacement. In this example the evidence of the blood loss and recovery is recorded by laboratory hematology data of Table 1. Table 1 shows hemoglobin record of a patient with severe blood loss and recovery.

TABLE 1

| Time | Hemoglobin | |
|---|---|---|
| (Days before sample) | g/dL | Comment |
| 28 | 13.5 | Patient's usual level |
| 23 | 9.3 | onset of bleed recorded |
| 17 | 8.6 | lowest blood level of hemoglobin |
| 1 | 11.0 | Recovery phase |
| 0 | 14.2 | back to normal |

It may be seen in the table that from the time of the lowest blood level the patient has replaced approximately 50% more red blood cells containing hemoglobin. Hence approximately 40% of the cells represented in FIG. 4 are less than 1 month in age, and the distortions and discontinuities in the chart are due to a discontinuity of the RBC age profile. On the day on which the sample is taken the reticulocyte count would be close to normal. However, according to the present invention the record of the excessive stimulus and response to RBC replacement has not been lost. A single sample contains the information representing the tabulated hematology data. Therapy of this patient for nutritional anemia resulted in a change in size from pathologically small RBC to normal size RBC. As a result this is an example of a rare condition in which the size of RBC is not completely in age order.

Like patterns of discontinuity may also be found in certain less common conditions, such as temporary kidney failure with uremia, where the production of RBC is temporarily altered, and then returns to normal, or with various forms of intermittent therapy of anemia, such as iron therapy or Erythropoietin.

### Example 5

### Making Conclusions from Regression Statistics Based in Population Distributions

Using the random sample results of Example 4, a variety of derived calculations are made with the approximately linear relationship with age and enzymatic activity. For example the point at which the curve intercepts the X axis in time is approximately 5 consecutive half life intervals in the cell aging process. For the enzyme Pyruvate kinase the published ½ life is 29 days in a limited population study. (Carol Seaman, et al “The decline of energetic Metablolism with aging Erythocytes and its relationship to cell death.” American Journal of Hematology 8:31-42 1980.) In data derived from time resolved fractions of individual patients this data may be calculated for each patient. in FIG. 5 shows that there is great variability in such individual patients but that the median extrapolated age is indeed 170 days, as predicted by a ½ life of 29 days. FIG. 6 shows a calculated theoretical series of ½ sequential life points. The prior art performed on average of many individuals is shown to agree with the individual patient calculation of the present invention.

In FIG. 5 a considerable presence of outliers brings attention to individual variability that patterns may be related to the condition of the individual patient. For example, the patients represented by the longest extrapolated PK activity all have the individual pattern of the patient represented in FIG. 3. One of these patients had compensated autoimmune hemolytic anemia as shown above, a second had no history, the third had long standing Renal Cell carcinoma, a condition known to produce excess secretion of erythropoietin. (Cohen et. Al. New Engl. J. Med 353:2477-2490, 2005). As a result the pattern is shown to be associated with EPO, and shows evidence for the value of the invention.

Although there are no athletes in the collected series of samples it is anticipated that self medication by athletes would produce patterns related to the amount and timing of self medication.

Further calculated distributions were made for the slope and correlation coefficient of the same population of 117 patient samples, shown in FIGS. 7 and 8. The outliers for the slope distribution are all patients with chronic anemia. Several of these patients had the anemia associated with long standing cancer. No status was available for the patients who are outliers on the correlation distribution. All of these patients had the pattern shown in FIG. 4, which is theoretically associated with a history of transfusion or intermittent therapy to increase red blood cell production.

It is understood that the invention can take advantage of further pairing of parameters. For example, there are other enzymes in RBC that are extremely stable such as 3-phosphglyceraldehyde dehydrogenase, having ½ lives of more than 240 days. These enzymes may be used as controls to be analyzed in the same sub-fractions, to provide a more reliable baseline for the number of cells being analyzed. Furthermore, once the principle of enzyme degradation with age is established there will be a search for other enzymes with rapid degradation in the RBC. The use of such enzymes in correlative evaluations for RBC conditions is incorporated in the present invention. In the same manner detection of paired evaluations with parameters having different rates of degradation and measured by other formats, including from cytometry and slide based cytometry are also part of the present invention.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method for analysis of red blood cell production dynamics, comprising:
a) obtaining a blood sample from a person;
b1) separating RBCs in the blood sample into ordered subsets of RBCs based on relative age-related physical parameter of the RBCs to obtain a first estimate of relative RBC age for each of the subsets of the RBCs, and obtaining a second estimate of the activity or concentration of a biomarker that degrades with RBC age within the subsets of the RBCs, and pairing the first estimate and the second estimate for each of the subsets to obtain paired estimates; or
b2) obtaining a pair of estimates simultaneously, the pair of the estimates comprising a first estimate and a second estimate, the first estimate being a measurement of the size of individual RBCs, the second estimate being a measurement of the activity or concentration of the biomarker, sorting the pair into ordered subsets of data based on the first estimate to obtain paired estimates;
c) generating a mathematical pattern or a graphical pattern based on the paired estimates or the ordered subsets of data; and
d) comparing the mathematical pattern or the graphical pattern with established patterns to determine the RBC production dynamics in the person.

2. The method of claim 1, wherein the separating step is performed by density centrifugation

3. The method of claim 2, wherein the one or more enzymes is at least one selected from the group consisting of pyruvate kinase or hexokinase.

4. The method of claim 1, further comprising:
identifying that the person has a particular condition that affects the RBC production dynamics thereof by comparing the generated pattern with established patterns that reflect the particular condition.

5. The method of claim 1, further comprising:
identifying that the person is rapidly producing RBCs immediately before the sample is obtained when the graphical pattern shows the second estimate for a young age RBC subset is higher than the remaining ordered subsets.

6. The method of claim 1, further comprising:
identifying that the person is rapidly producing RBCs before the sample is obtained when the graphical pattern shows the second estimate for a relatively older age RBC subset is higher than the remaining ordered subsets.

7. The method of claim 1, further comprising:
identifying that the person has a high blood level of erythropoietin as compared with a normal person when the graphical pattern shows the second estimates remains high.

8. The method of claim 1, further comprising:
identifying that the person has an abnormal hematological condition when the mathematical pattern falls outside the range of a normal statistical population distribution of mathematical patterns that are generated from a collection of slopes, intercepts and/or correlation coefficients of the paired estimates.

9. The method of claim 1, wherein the step of obtaining the pair of estimates simultaneously is performed by flow cytometry.

10. The method of claim 1, further comprising:
identifying that the person receives an RBC supplementation before the blood sample is obtained when the graphical pattern exhibits more than one discontinuity and the second estimates remains low or normal.

11. The method as in claim 10, wherein the RBC supplementation is autologous blood transfusion.
